(19) United States
(12) Patent Application Publication
(10) Pub. No.: US 2009/0036778 A1
(43) Pub. Date: Feb. 5, 2009

(54) DUAL FREQUENCY DOPPLER ULTRASOUND PROBE

(75) Inventors: Martin L. Cohen, Newport, RI (US); John Haefele, Putnam, CT (US); Anthony Castillo, Cranston, RI (US)

Correspondence Address: BARLOW, JOSEPHS & HOLMES, LTD., 101 DYER STREET, 5TH FLOOR, PROVIDENCE, RI, 02903, US

(73) Assignee: UNETIXS VASCULAR INCORPORATED, North Kingstown, RI (US)

(21) Appl. No.: 11/949,067

(22) Filed: Dec. 3, 2007

Related U.S. Application Data
(60) Provisional application No. 60/953,014, filed on Jul. 31, 2007.

(51) Int. Cl. A61B 8/00

(52) U.S. Cl. 600/459

(57) ABSTRACT

An ultrasonic Doppler probe is provided for use in connection with non-invasive Doppler imaging of fluid flow within the human body. The Doppler probe can be selectively operated at more than one frequency during the course of a Doppler imaging examination thereby enhancing the resolution of the image obtained while also increasing the effective depth of the image. The probe of the present invention employs piezo-electric materials for the formation of acoustic transmitting and receiving transducers that are positioned within the probe to allow the probe to be selectively operated at a number of different frequencies spanning no more than one octave in frequency range.

### CROSS-REFERENCE TO RELATED APPLICATIONS

This application is related to and claims priority from earlier filed U.S. Provisional Patent Application No. 60/953,014, filed Jul. 31, 2007.

### BACKGROUND OF THE INVENTION

The present invention relates generally to an ultrasonic probe for non-invasive measurement of fluid flow within the human body. More specifically, the present invention relates to an ultrasonic Doppler probe for measuring fluid flow within the human body that incorporates a dual frequency acoustical transducer, thereby allowing operation of the probe at both higher and lower frequencies without the need for the operator to change probes.

As ultrasonic technology has improved, non-invasive ultrasonic diagnostic equipment has become an indispensable tool for clinical use. For many years, real-time B-mode ultrasound imagers have been used in connection with the investigation and imaging of stationary soft tissue structures within the human body. In addition, the more recent development of Doppler ultrasound scanners has facilitated the non-invasive investigation of moving fluids within the human body. In fact, Doppler ultrasound has become the standard in available techniques for non-invasively detecting and measuring the velocity of moving structures within the human body, and particularly to provide a real time estimate of the blood velocity traveling at various points within the body.

The basic scientific principal underlying Doppler ultrasonography is based on the fact that ultrasonic waves, when directed at a moving object, undergo a frequency shift upon reflection and/or scattering by that object. Generally, the magnitude and the direction of the frequency shift in turn provides information regarding the motion of the object being observed. In other words, the magnitude of the frequency change is dependent upon how fast the object is moving. In this context, there are several different depictions of blood flow that are produced through medical Doppler imaging, including color flow imaging, power Doppler and spectral sonograms. Color flow imaging (CFI), is employed for imaging a whole region of the body and displays a real-time image of mean velocity distribution. CFI provides an estimate of the mean velocity of flow with a vessel by color coding the information and displaying it, super positioned on a dynamic B-mode image or black and white image of anatomic structure. While CFI displays the mean or standard deviation of the velocity of observed objects, such as the blood cells, in the given region, power Doppler (PD) in contrast displays a measurement of the amount of moving objects in the area. A PD image is an energy image wherein the energy of the flow signal is displayed. Thus, PD depicts the amplitude or power of the Doppler signals rather than the frequency shift. This allows detection of a larger range of Doppler shifts and thus better visualization of small vessels. In all of these technologies, however, the images produced show only the direction of flow and do not provide any no velocity information. Finally, spectral Doppler or spectral sonogram utilizes a pulsed wave system to interrogate a single range gate or sampling volume and displays the velocity distribution as a function of time.

It is also of note that in the prior art, Doppler imaging is done using different acoustical frequencies, where the selection of acoustical frequency is a compromise between resolution and the ability to perceive the internal structure being imaged. This compromise is based generally on the fact that while higher frequency Doppler waves provide higher resolution they do not penetrate into the body as deeply, lower frequencies penetrate more deeply but the penetration depth is achieved at the expense of resolution. A processor is then employed to receive the electrical signals from the Doppler probe and operate upon them to determine the information that is to be provided to the user on the display. In some systems, the processor generates an electrical signal that is converted and translated in the probe as an acoustic signal, while in other systems the probe itself generates the signal to be transmitted. Similarly, in some systems, the probe simply converts the received acoustic signal to an electrical signal that is transferred to the processor while in others, the probe processes the electrical form of received acoustic signal so that it at a different (lower) frequency and then provides the converted data to the processor.

The difficulty that is encountered in the prior art is that the currently available ultrasound probes operate at only a single frequency. As a result the operator must change probes to employ a different acoustical frequency for a portion of the examination. Accordingly, there is a need for a single ultrasonic probe that can be selectively operated at more than one frequency, thereby eliminating the need for the operator to switch probes during the investigation process.

### BRIEF SUMMARY OF THE INVENTION

In this regard, the present invention provides for a Doppler probe that can be selectively operated at more than one frequency during the course of a Doppler imaging examination. The probe of the present invention employs piezo-electric materials for the formation of acoustic transmitting and receiving transducers that are positioned within the probe to allow the probe to be operated at a number of different frequencies spanning no more than one octave in frequency range.

In one embodiment the probe of the present invention includes an acoustic transducer, a receiver and an operator control switch to selectively to select the frequency of operation from either of two predetermined frequencies and to show which frequency of operation is being used.

In an alternate embodiment the switching function is transferred from the probe and implemented via a processor based control selector.

In another alternate embodiment the transmitting and receiving components are provided in the processor so that the probe itself essentially contains only the acoustic transducer and the probe accepts a high frequency electrical signal from the processor for acoustic transmission and the probe provides the processor with the high frequency signal received by the receiving section of the acoustic transducer.

In yet another alternate embodiment, the signals obtained by the receiving section of the acoustic transducer are converted to digital form by an analog-to-digital converter (A/D) and the resulting digital information is transferred to the processor for further processing such as complex demodulation and Doppler frequency extraction.

In still a further alternate embodiment, a self-contained probe is provided that includes a wireless interface and a battery in order to provide its own power. The probe converts the received signals to a digital signal that is transmitted via the wireless interface to the processor.

It is therefore an object of the present invention to provide a probe assembly for use in connection with ultrasonic Doppler imaging, which includes acoustical transducers therein that allow selective operation across at least two different frequencies. It is a further object of the present invention to provide a probe for use in ultrasonic Doppler imaging that includes acoustical transmitter and receiver components capable of selectively operating across at least two distinct frequencies while transmitting the information collected by the receiver to a processing device. It is still a further object of the present invention to provide a self contained probe for use in ultrasonic Doppler imaging that can be selectively operated across at least two distinct frequencies while wirelessly transmitting the information collected by the receiver to a processing device.

These together with other objects of the invention, along with various features of novelty that characterize the invention, are pointed out with particularity in the claims annexed hereto and forming a part of this disclosure. For a better understanding of the invention, its operating advantages and the specific objects attained by its uses, reference should be had to the accompanying drawings and descriptive matter in which there is illustrated a preferred embodiment of the invention.

### BRIEF DESCRIPTION OF THE DRAWINGS

In the drawings which illustrate the best mode presently contemplated for carrying out the present invention:

FIG. 1 is a schematic depiction of an ultrasonic probe in accordance with the teachings of the present invention;

FIG. 2 is a schematic depiction of the ultrasonic probe of FIG. 1 with additional operational components depicted;

FIG. 3 is a schematic depiction of a first alternate embodiment ultrasonic probe in accordance with the teachings of the present invention;

FIG. 4 is a schematic depiction of a second alternate embodiment ultrasonic probe in accordance with the teachings of the present invention;

FIG. 5 is a schematic depiction of a third alternate embodiment ultrasonic probe in accordance with the teachings of the present invention; and

FIG. 6 is a schematic depiction of a fourth alternate embodiment ultrasonic probe in accordance with the teachings of the present invention.

### DETAILED DESCRIPTION OF THE INVENTION

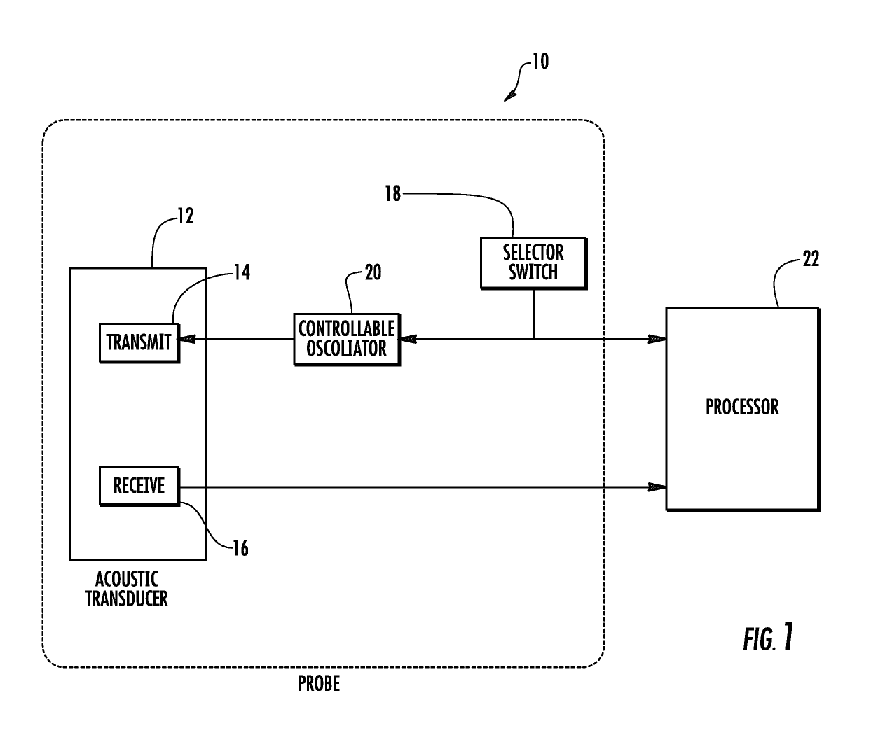

Now referring to the drawings, a schematic depiction of the ultrasonic probe of the present invention is shown and generally illustrated at 10 in FIG. 1. As was stated above, the present invention is directed at providing an ultrasonic probe 10 that is selectively operable over at least two different frequencies, thereby allowing an operator to conduct an ultrasonic examination across differing ultrasonic frequencies without having to change probes. In this regard, in a preferred embodiment the probe 10 of the present invention generally includes an acoustic transducer 12 having a transmit section 14 that creates and transmits an acoustic signal from a high frequency electrical signal and a receive section 16 that receives a reflection of the transmitted acoustic signal and converts the received reflection into an electrical signal. Further, the probe 10 includes a selection switch 18 that allows the user to selectively determine a frequency at which the acoustic signal is transmitted.

As will be appreciated by one skilled in the art, the transmit section 14 in the acoustical transducer 12 is formed from a piezo-electric material that vibrates in response to electrical signals, thereby generating sound waves corresponding to the electrical signal. In this regard, a driver in the form of an oscillator 20 is used to generate a high frequency electrical signal having a wavelength that corresponds to the frequency at which the transmitter 14 in the transducer 12 is to be operated. In other words, the oscillator 20 generates a high frequency electrical signal that causes the piezo-electric material in the transmitter 14 to vibrate thereby emitting ultrasonic waves. In contrast to the prior art, the present invention employs a controllable oscillator 20 that generates a selectively variable frequency electrical signal in response to the frequency selection switch 18. As a result, with the frequency selection switch 18 in a first position, the controllable oscillator 20 generates a first electrical signal that in turn drives the transmit section 14 of the acoustic transducer 12 at a first frequency. When the selection switch 18 is moved to a second position, the controllable oscillator 20 generates a second electrical signal that in turn drives the transmit section 14 of the acoustic transducer 12 at a second frequency. Further, the selector switch 18 also provides a signal to a processor 22 with which the ultrasonic probe 10 is interfaced thereby alerting the processor 22 to the frequency at which the acoustical transducer 12 is operating. This information is necessary so that the processor 22 can properly interpret the signal being transmitted by the transmit section 14 and returned by the receiver section 16, so that it can display the frequency in use to the operator and so that it can include the information regarding the frequency being used in the data record of the test.

In this regard, the probe 10 of the present invention includes an acoustical transducer 12 that can be selectively operated at a variety of different frequencies thereby allowing a comprehensive Doppler examination to be performed without the need for switching between multiple probes. Preferably, the range of multiple frequencies is limited to a range that falls into a single octave range. For example, the probe 10 can be selectively operated at the pair of frequencies of 5 MHz and 8 MHz or the pair of frequencies of 2.1 MHz and 3.9 MHz.

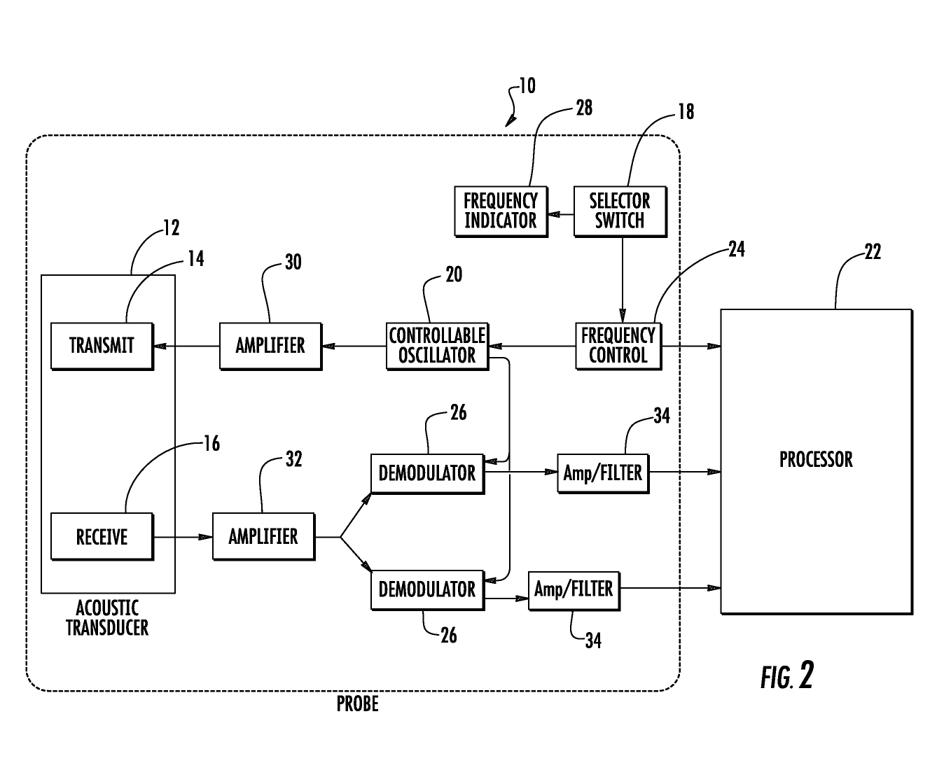

Turning now to FIG. 2, in addition to including the above described elements, the probe 10 of the present invention preferably includes a frequency controller 24 that interprets the input from the frequency selection switch 18 to select and change the signal that is being generated by the controllable oscillator 20 In this regard, the frequency controller 24 serves to control the controllable oscillator 20 by providing a drive signal to the controllable oscillator 20 that in turn generates and transmits a high frequency electrical signal to the transmitter 14 in the acoustical transducer 12. The controllable oscillator 20 also provides a signal to the signal demodulator 26 on the receiver side 16 of the probe 10 in order to allow the demodulator 26 to correctly interpret the signals received from the receiver 16. The selector switch 18 may also send a signal to a frequency indicator 28 such as a lamp, an LED or an LCD display that visually shows the operator which operational frequency has been selected. The probe 10 of the present invention may also include a transmit amplifier 30 to amplify the electrical signal generated by the controllable oscillator 20 before passing it along to the transmitting section 14 of the acoustic transducer 12 and a receiving amplifier 32 to accept the signal from the receiving section 16 of the acoustic transducer 12 and amplify it for further processing. Further, the probe 10 may include an I-Q demodulator 26 and filters 34 to translate the received signal to a complex baseband form in order to perform Doppler processing within the processor 22.

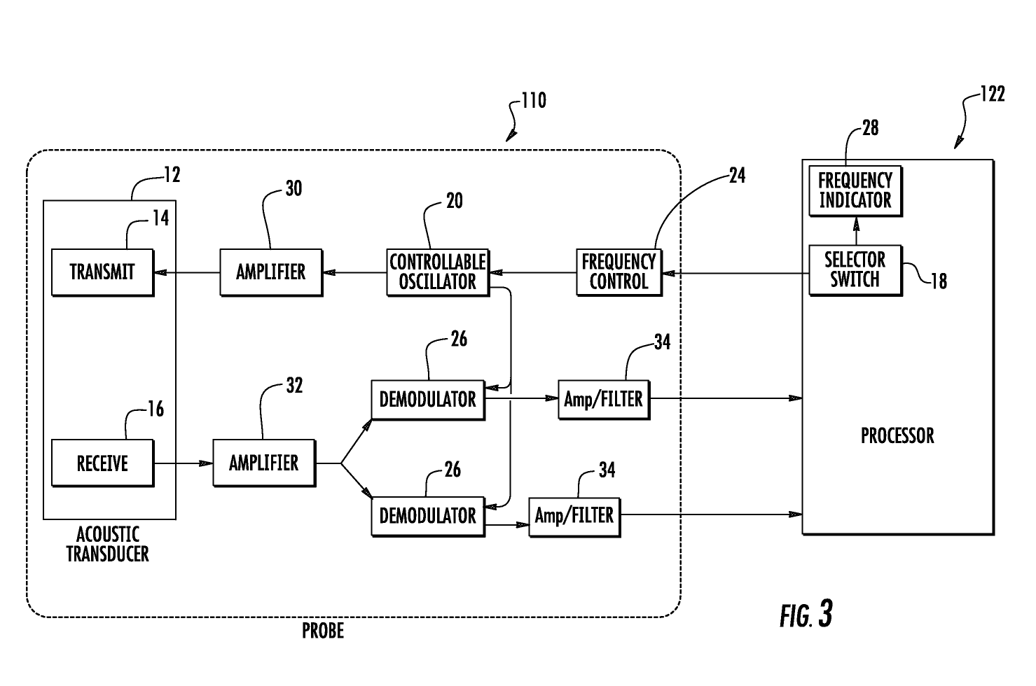

In addition to the embodiment detailed above, there are a number of possible alternative embodiments of the present invention. In a first alternative embodiment, as depicted in FIG. 3, the functions of the frequency selector switch 18 and the frequency indicator 28 are removed from the probe 110 and implemented in the processor 122. The frequency selection may in this embodiment be effectuated by a physical selector switch 18 or may be software implemented. The signal instructing the controllable oscillator 20 which one of the two predetermined frequencies to use is then is provided by the processor 122 by to the probe 110.

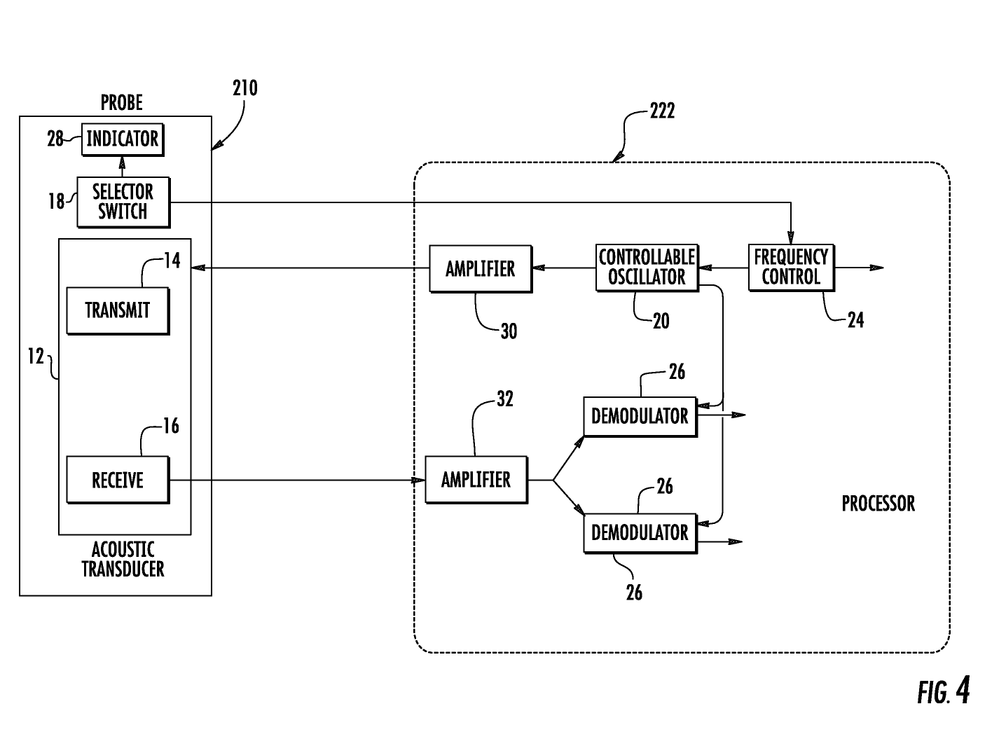

In a second alternative embodiment, depicted at FIG. 4, the probe 210 only contains the acoustic transducer 12 while the remaining transmit and receiving components, or major portions thereof, are relocated to the processor 222. In this embodiment, the probe 210 itself essentially contains only the acoustic transducer 12 with the receiving section 16 and the transmit section 14. The probe 210 accepts a high frequency electrical signal from the controllable oscillator 20, which in this embodiment is located within the processor 222, via the amplifier 30. In response to the signal from the controllable oscillator 20, the transmitter 14 generates an acoustic transmission that is in turn received in the receiver 16 and is provided to the processor 222 as a high frequency signal. In this alternative implementation, while the selector switch 18 and frequency indicator 28 are depicted as being provided within the probe 210, clearly the selector switch 18 and frequency indicator 28 may be provided in the processor 222 as well as described above with regard to the earlier embodiment in FIG. 3.

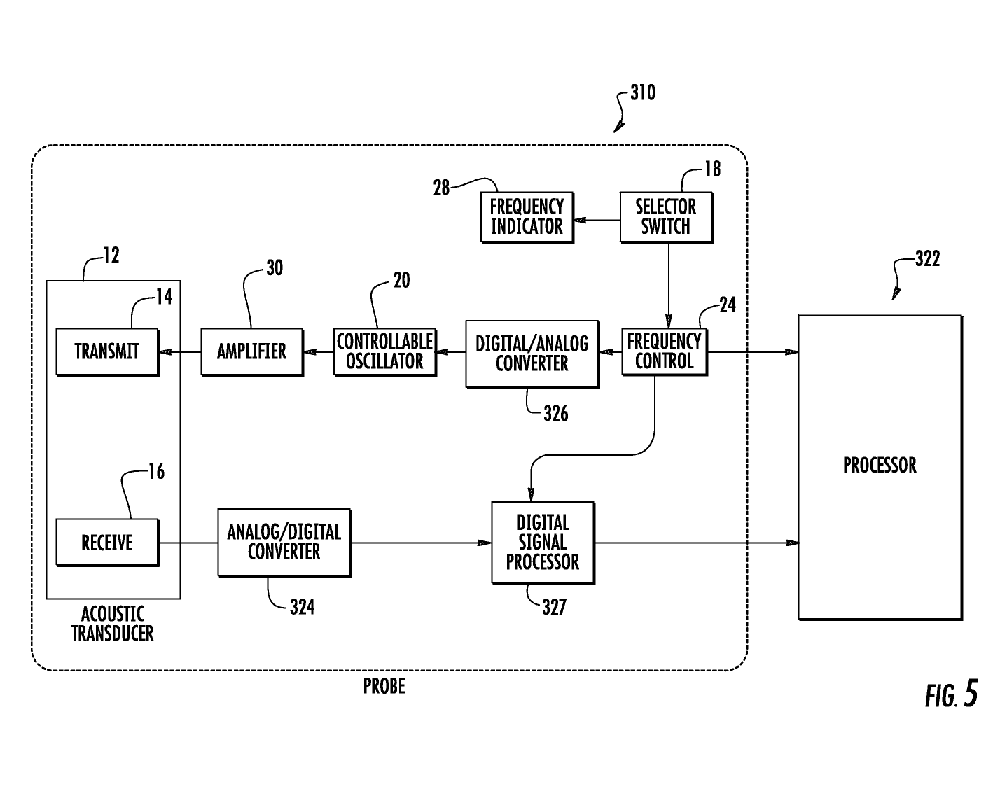

FIG. 5 depicts a third alternative embodiment wherein communication between the probe 310 and the processor 322 is effectuated via digital communication signals. The signals received at the receiving section 16 of the acoustic transducer 12 are converted into a digital signal using an analog-to-digital converter (A/D) 324 and the resulting digital information is transferred to the processor 322 for further processing such as complex demodulation and Doppler frequency extraction. Alternatively, the probe 310 may contain a digital signal processor 327 that performs some of the latter processing steps, thereby lowering the data rate of the information to be transferred to the processor 322. In such cases, the digital signal processor 327 receives information on the frequency in use from the frequency controller 24. On the transmit side, the frequency controller 24, controllable oscillator 20, selector switch 18, frequency indicator 28 and transmit amplifier 30 may be contained in the probe 310 as shown. Further, any portion of these components may also be contained within the processor 322 as described above at FIG. 4. In any case, in this embodiment, a digital signal is generated by the frequency controller 24 that is then transmitted to a digital-to-analog converter (D/A) 326 where the digital signal is processed into an analog signal for use by the controllable oscillator 20 in generating the transmit signal. In all other respects the present embodiment operates as described above in the wholly analog embodiments.

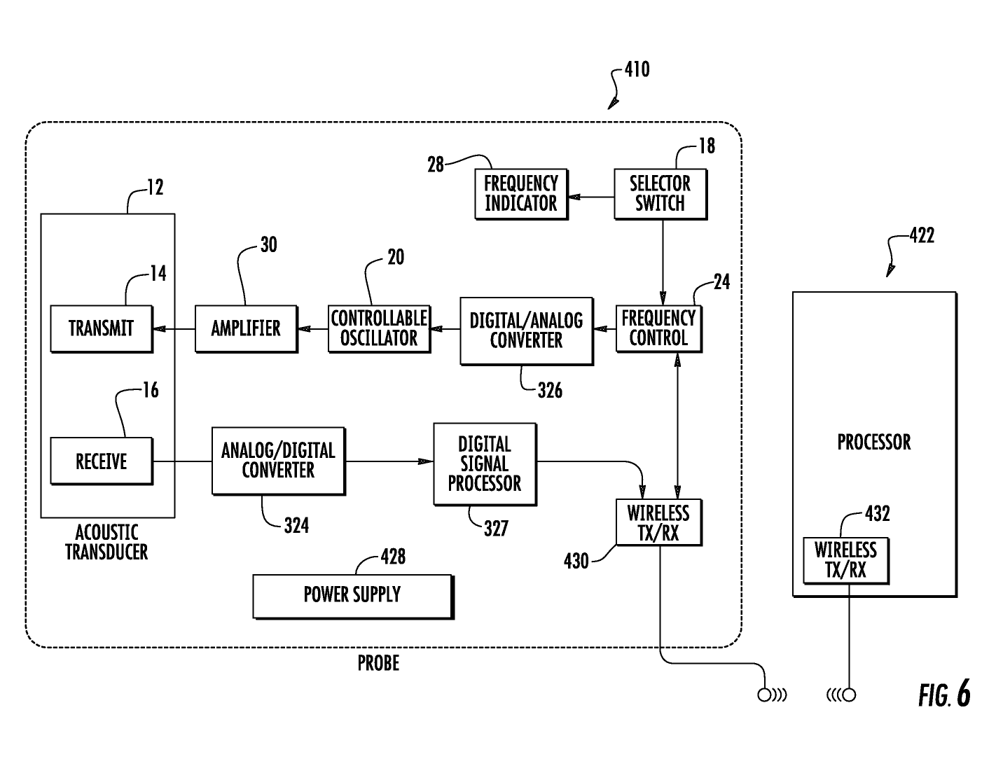

Finally, in a fourth alternative embodiment depicted at FIG. 6, a wireless self-contained probe 410 in accordance with the teachings of the present invention is provided. In this embodiment, in addition to the features described in the third alternate embodiment at FIG. 5 above, the probe 410 also includes a power source 428 therein such as a battery. Further, the probe 410 includes a wireless digital interface transmit/receive module 430 that communicates with a corresponding wireless transmit/receive module 432 in the processor 422 thereby eliminating the need for cabling between the probe 410 and the processor 422. This allows wireless digital communication between the prove 410 and the processor 422. In this embodiment, it is preferred that all of the analog components be positioned on the probe 410 thereby requiring that only digital signals be transmitted wirelessly.

It should be appreciated that in the scope of the present invention the important point of novelty is that the probe assembly allows operation over at least two different signal frequencies without requiring that the user switch probes. In this regard, it can therefore be seen that the present invention provides a novel and useful ultrasonic probe assembly that enhances the operator's ability to perform non-invasive ultrasonic examinations while enhancing the overall image obtained and reducing the time required to obtain a high quality image. By allowing the operator to selectively operate at multiple frequencies, Doppler images can be obtained that have both improved resolution with an increased depth of penetration within the human body. For these reasons, the instant invention is believed to represent a significant advancement in the art, which has substantial commercial merit.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed:

1. An ultrasonic probe comprising:
an acoustical transducer having a transmit section capable receiving and converting a high frequency electrical signal to an ultrasonic sound wave;
an oscillator in electrical communication with said acoustical transducer, said oscillator configured to selectively generate and transmit at least first and second high frequency electrical signals to said acoustical transducer; and
a selector switch having at least a first position and a second position, said selector switch in electrical communication with said oscillator, wherein said selector switch in said first position causes said oscillator to generate and transmit said first high frequency electrical signal and said selector switch in said second position causes said oscillator to generate and transmit said second high frequency electrical signal.

2. The ultrasonic probe of claim 1, further comprising:
a processor in electrical communication with said acoustical transducer, said oscillator and said selector switch, wherein said selector switch provides a signal to said processor to indicate the frequency at which said oscillator is operating.

3. The ultrasonic probe of claim 1, further comprising:
a frequency indicator in electrical communication with said selector switch, said frequency indicator providing a visual representation to indicate the frequency at which said oscillator is operating.

4. The ultrasonic probe of claim 1, further comprising:
a receive section in said acoustic transducer capable receiving and converting an ultrasonic sound wave to a high frequency electrical signal;
a demodulator in electrical communication with said receive section and said oscillator that converts said high frequency electrical signal to a lower frequency I-Q electrical signal; and
a frequency control in electrical communication with said selector switch and said oscillator, said frequency control interpreting input from said selector switch to generate a drive signal that is transmitted to said oscillator.

5. An ultrasonic imaging device for non-invasive imaging of a target within a human body comprising:
a probe including an acoustical transducer having a transmit section capable of selectively receiving and converting at least two different high frequency electrical signals to an ultrasonic sound wave for transmission at said target and a receive section capable receiving a reflection of said ultrasonic sound wave and converting said reflection to a high frequency electrical signal;
a processor in electrical communication with said acoustical transducer, wherein said processor generates an image of said target based on said high frequency electrical signal generated by said receive section.

6. The ultrasonic imaging device of claim 5, the probe further comprising:
a selector switch having at least a first position and a second position, said selector switch in electrical communication with said acoustic transducer, wherein said selector switch in said first position causes said acoustic transducer to generate and transmit said first high frequency electrical signal and said selector switch in said second position causes said acoustic transducer to generate and transmit said second high frequency electrical signal.

7. The ultrasonic imaging device of claim 6, the probe further comprising:
a frequency indicator in electrical communication with said selector switch, said frequency indicator providing a visual representation to indicate the frequency at which said acoustic transducer is operating.

8. The ultrasonic imaging device of claim 6, the probe further comprising:
an oscillator in electrical communication with said transmit section, said oscillator configured to selectively generate and transmit first and second high frequency electrical signals to said transmit section;
a demodulator in electrical communication with said receive section and said oscillator; and
a frequency control in electrical communication with said selector switch and said oscillator, said frequency control interpreting input from said selector switch to generate a drive signal that is transmitted to said oscillator and to said demodulator.

9. The ultrasonic imaging device of claim 6, the processor further comprising:
an oscillator in electrical communication with said transmit section, said oscillator configured to selectively generate and transmit first and second high frequency electrical signals to both said transmit section and said demodulator;
a demodulator in electrical communication with said receive section and said oscillator; and
a frequency control in electrical communication with said selector switch, said oscillator and said demodulator, said frequency control interpreting input from said selector switch to generate a drive signal that is transmitted to said oscillator.

10. The ultrasonic imaging device of claim 5, the processor further comprising:
a selector switch having at least a first position and a second position, said selector switch in electrical communication with said acoustic transducer, wherein said selector switch in said first position causes said acoustic transducer to generate and transmit said first high frequency electrical signal and said selector switch in said second position causes said acoustic transducer to generate and transmit said second high frequency electrical signal.

11. The ultrasonic imaging device of claim 10, the processor further comprising:
a frequency indicator in electrical communication with said selector switch, said frequency indicator providing a visual representation to indicate the frequency at which said acoustic transducer is operating.

12. The ultrasonic imaging device of claim 10, the processor further comprising:
an oscillator in electrical communication with said transmit section and said demodulator, said oscillator configured to selectively generate and transmit first and second high frequency electrical signals to said transmit section and said demodulator;
a demodulator in electrical communication with said receive section and said oscillator; and
a frequency control in electrical communication with said selector switch, said oscillator and said demodulator, said frequency control interpreting input from said selector switch to generate a drive signal that is transmitted to said oscillator.

13. An ultrasonic imaging device for non-invasive imaging of a target within a human body comprising:
a probe including a wireless transmit/receive module and an acoustical transducer having a transmit section capable of selectively receiving and converting at least two different high frequency electrical signals to an ultrasonic sound wave for transmission at said target and a receive section capable receiving a reflection of said ultrasonic sound wave and converting said reflection to a high frequency electrical signal;
a processor including a wireless transmit/receive module in wireless electrical communication with said acoustical transducer, wherein said processor generates an image of said target based on said high frequency electrical signal generated by said receive section.

14. The ultrasonic imaging device of claim 13, the probe further comprising:
a selector switch having at least a first position and a second position, said selector switch in electrical communication with said acoustic transducer, wherein said selector switch in said first position causes said acoustic transducer to generate and transmit said first high frequency electrical signal and said selector switch in said second position causes said acoustic transducer to generate and transmit said second high frequency electrical signal.

15. The ultrasonic imaging device of claim 14, the probe further comprising:
a frequency indicator in electrical communication with said selector switch, said frequency indicator providing a visual representation to indicate the frequency at which said acoustic transducer is operating.

16. The ultrasonic imaging device of claim 14, the probe further comprising:
an oscillator in electrical communication with said transmit section and said demodulator, said oscillator configured to selectively generate and transmit first and second high frequency electrical signals to said transmit section and said demodulator;
an analog to digital converter in electrical communication with said receive section;
a frequency control in electrical communication with said selector switch and said oscillator, said frequency control interpreting input from said selector switch to generate a drive signal that is transmitted to said oscillator; and
a digital to analog converter to convert digital signals from said processor to analog signals usable by said frequency control.

17. The ultrasonic imaging device of claim 16 wherein said probe and said processor wirelessly exchange signals in a digital format.
